(19) United States
(12) Patent Application Publication
(10) Pub. No.: US 2012/0130166 A1
(43) Pub. Date: May 24, 2012

(54) LIGHT-EMITTING APPARATUS AND ENDOSCOPE HAVING LIGHT-EMITTING APPARATUS

(75) Inventors: Yoshiro NISHIMURA, Okaya-shi (JP); Hiroshi SUZUSHIMA, Kamiina-gun (JP)

(73) Assignee: OLYMPUS CORPORATION, Tokyo (JP)

(21) Appl. No.: 13/295,530

(22) Filed: Nov. 14, 2011

(30) Foreign Application Priority Data
Nov. 19, 2010 (JP) 2010-259323

(51) Int. Cl. A61B 1/04; H01L 33/50

(52) U.S. Cl. 600/109; 257/89; 257/E33.061

(57) ABSTRACT

A light-emitting apparatus includes a blue color LED, a fluorescent substance mixed transparent resin that covers the blue color LED and generates yellow light, a red LED disposed on the fluorescent substance mixed transparent resin and a transparent resin that covers the red LED.

### CROSS REFERENCE TO RELATED APPLICATION

This application claims the benefit of Japanese Application No. 2010-259323 filed in Japan on Nov. 19, 2010, the contents of which are incorporated herein by this reference.

### BACKGROUND OF THE INVENTION

1. Field of the Invention

Embodiments of the present invention relate to a light-emitting apparatus having a plurality of light-emitting elements and an endoscope having the light-emitting apparatus.

2. Description of the Related Art

Light-emitting apparatuses using semiconductor light-emitting elements are small and deliver high power efficiency. For this reason, light-emitting apparatuses having semiconductor light-emitting elements such as a light-emitting diode (LED) or a laser diode (LD) are used as various light sources. Light generated by a semiconductor light-emitting element has a steep spectrum distribution. For this reason, for example, a light-emitting apparatus that generates white color light needs to use a plurality of semiconductor light-emitting elements that generate light having different wavelengths.

Japanese Patent Application Laid-Open Publication No. 2008-130777 discloses a light-emitting apparatus 101 that generates so-called white color light of a mixture of three wavelengths combining a blue color LED 111, a green color LED 113 and a red color LED 115. As shown in FIG. 1, the light-emitting apparatus 101 includes a wiring board 110, the blue color LED 111 die-bonded onto the wiring board 110, the green color LED 113 bonded onto the blue color LED 111 via a transparent resin 112, and further the red color LED 115 bonded onto the green color LED 113 via a transparent resin 114.

### SUMMARY OF THE INVENTION

A light-emitting apparatus according to an embodiment of the present invention includes a first light-emitting element that generates light of a first wavelength band, a first sealing resin that covers the first light-emitting element, the first sealing resin being made of a transparent resin mixed with a first fluorescent substance that converts the light of the first wavelength band to light of a third wavelength band having a longer wavelength, a second light-emitting element disposed on the first sealing resin and generating light of a second wavelength band and a second sealing resin made of a transparent resin that covers the second light-emitting element.

### BRIEF DESCRIPTION OF THE DRAWINGS

FIG. 1 is a diagram illustrating a cross-sectional structure of a conventional light-emitting apparatus;

FIG. 2 is a diagram illustrating a cross-sectional structure of a light-emitting apparatus according to a first embodiment;

FIG. 3 is a diagram illustrating light-emitting wavelengths of the light-emitting apparatus of the first embodiment;

FIG. 4 is a diagram illustrating a cross-sectional structure of a light-emitting apparatus according to a second embodiment;

FIG. 5 is a diagram illustrating light-emitting wavelengths of the light-emitting apparatus of the second embodiment;

FIG. 6 is a diagram illustrating light-emitting wavelengths of the light-emitting apparatus of the second embodiment;

FIG. 7 is a diagram illustrating light-emitting wavelengths of the light-emitting apparatus of the second embodiment; and

FIG. 8 is a diagram illustrating an endoscope according to a third embodiment.

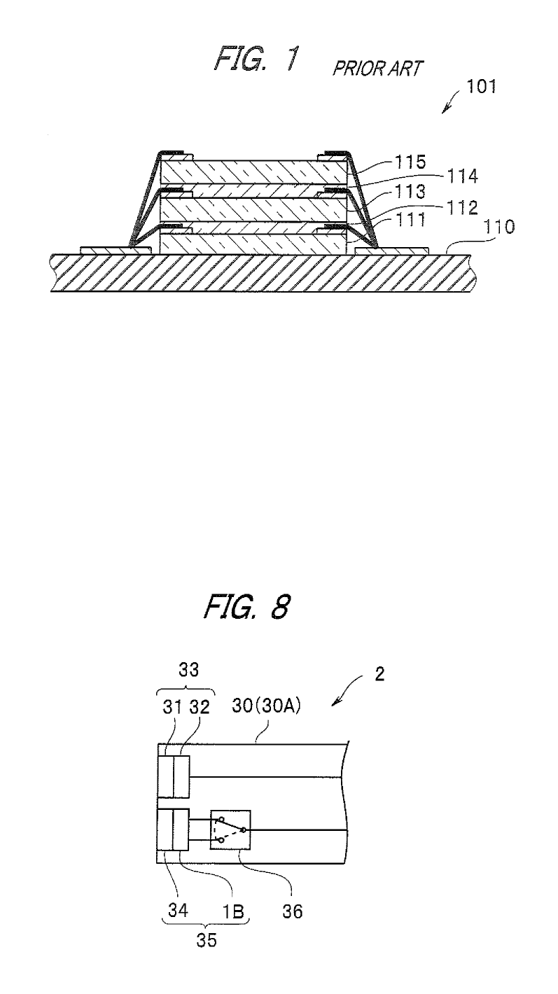

### DETAILED DESCRIPTION OF THE PREFERRED EMBODIMENTS

### First Embodiment

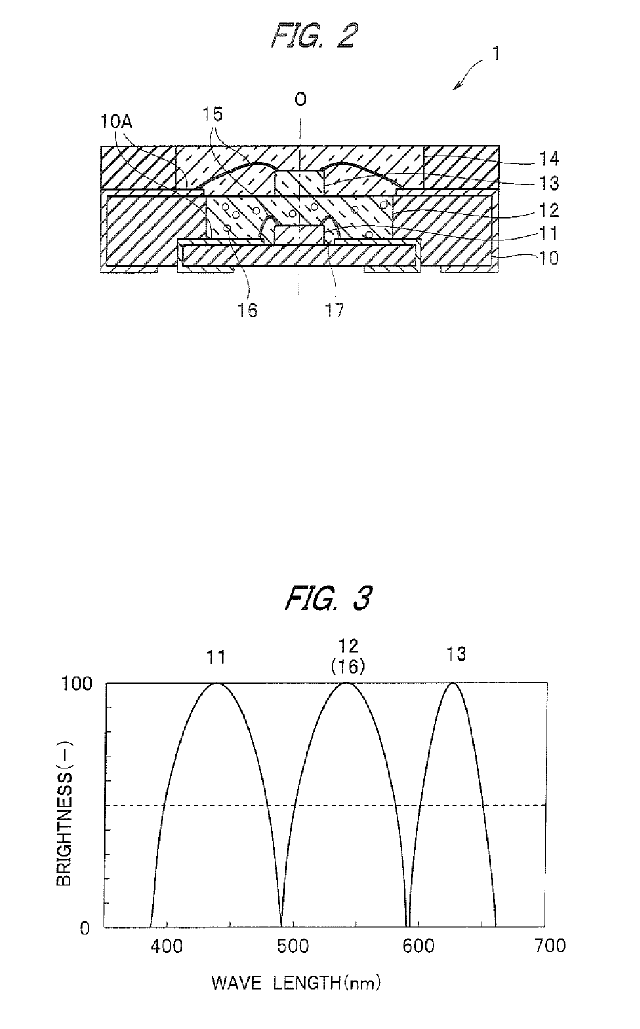

FIG. 2 is a diagram illustrating a cross-sectional structure of a light-emitting apparatus 1 according to a first embodiment. The following drawings are all schematic views and a vertical-horizontal ratio or the like is different from the actual one.

As shown in FIG. 2, the light-emitting apparatus 1 includes a substrate 10, a blue color LED 11 which is a first light-emitting element, a fluorescent substance mixed transparent resin (hereinafter also referred to as “fluorescent resin”) 12 which is a first sealing resin, a red color LED 13 which is a second light-emitting element and a transparent resin 14 which is a second sealing resin. The blue color LED 11 and the red color LED 13 are disposed on substantially a same optical axis line O.

The blue color LED 11 is die-bonded onto the substrate 10 and the fluorescent resin 12 covers and seals the blue color LED 11. The red color LED 13 is die-bonded onto the fluorescent resin 12 and the transparent resin 14 covers and seals the red color LED 13. That is, the fluorescent resin 12 covers not only a top surface but also sides of the blue color LED 11 and the transparent resin 14 covers not only a top surface but also sides of the red color LED 13.

As shown in FIG. 3, the blue color LED 11 emits purple to blue light having a wavelength of 395 nm to 480 nm and the red color LED 13 emits red light having a wavelength of 600 nm to 650 nm. The fluorescent resin 12 is made of a transparent resin 12B which is mixed with a fluorescent substance 12A that generates yellow light having a longer wavelength of 500 nm to 580 nm upon receiving blue light. The light-emitting wavelength is represented by a full width at half maximum wavelength.

As the light-emitting element, an LED is preferable, but a semiconductor light-emitting element such as LD can produce similar effects, and moreover, an organic EL element may also be used. As the fluorescent substance 12A, minute particles such as YAG (yttrium aluminum garnet)-based fluorescent substance or TAG (terbium aluminum garnet)-based fluorescent substance may be used.

The substrate 10 is made of ceramic, glass, aluminum nitride, aluminum, copper, glass fabric base epoxy, polyimide or the like and may also be a package having a package function.

The method of die-bonding the blue color LED 11 and the red color LED 13 may be realized using a transparent resin adhesive, a white color resin adhesive, an Ag paste, a eutectic solder or the like.

A bonding pad (not shown) of the blue color LED 11 and the red color LED 13 is electrically connected to a bonding lead 10A of the substrate 10 via a bonding wire 15 made of a fine metal wire of Au, Al, Cu or the like.

Electric connections between the blue color LED 11, the red color LED 13 and the substrate 10 may be realized according to a flip chip scheme or TAB (Tape Automated Bonding) scheme. As the transparent resin 12B and the transparent resin 14, an epoxy-based resin, a silicone-based resin, an acrylic-based resin or the like is used. As the fluorescent resin 12, a fluorescent substance mixed flat plate mixed with the fluorescent substance 12A may be used. Furthermore, after sealing the blue color LED 11/red color LED 13 with a transparent resin, the fluorescent substance mixed flat plate may be bonded onto the transparent resin. As the transparent flat plate, a glass plate, a quartz plate, a sapphire plate, an aluminum nitride plate, a transparent resin plate or the like may be used.

In the case of the aforementioned conventional light-emitting apparatus 101 with the three-layered light-emitting elements, light emitted from the light-emitting elements attenuates due to influences of the transmittance of the light-emitting elements and the transparent resin located thereon, and therefore high luminance may not be easily achieved.

By contrast, although the light-emitting apparatus 1 is provided with only two-layered light-emitting elements, the light-emitting apparatus 1 generates high luminance white color light of a mixture of three wavelengths; purple to blue light of wavelength 395 nm to 480 nm, yellow light of wavelength 500 nm to 580 nm and red light of wavelength 600 nm to 650 nm.

### Modification Example of First Embodiment

In a combination of the first light-emitting element, the fluorescent substance 12A of the fluorescent resin 12 and the second light-emitting element, the light-emitting wavelength of the fluorescent substance 12A needs only to be longer than the light-emitting wavelength of the first light-emitting element.

For example, consider a combination of the light-emitting wavelength of the first light-emitting element of 395 to 480 nm (purple to blue), the light-emitting wavelength of the fluorescent substance 12A of 600 nm to 650 nm (red) and the light-emitting wavelength of the second light-emitting element of 500 nm to 580 nm (green to yellow).

Furthermore, it is also possible to use another combination; the light-emitting wavelength of the first light-emitting element of 500 nm to 580 nm (green to yellow), the light-emitting wavelength of the fluorescent substance 12A of 600 nm to 650 nm (red) and the light-emitting wavelength of the second light-emitting element of 395 to 480 nm (purple to blue), for example.

A layering order of the fluorescent resin 12 and the transparent resin 14 may be reversed. That is, the first light-emitting element may be sealed with the transparent resin 14 and the second light-emitting element may be sealed with the fluorescent resin 12. In that case, suppose the light-emitting wavelength of the fluorescent substance 12A is longer than the wavelength(s) of both or one of the first light-emitting element and the second light-emitting element. For example, consider a combination of the light-emitting wavelength of the first light-emitting element of 600 nm to 650 nm (red), the light-emitting wavelength of the second light-emitting element of 395 to 480 nm (purple to blue) and the light-emitting wavelength of the fluorescent substance 12A of the fluorescent resin 12 on the second light-emitting element of 500 nm to 580 nm (green to yellow).

An optical part such as lens/prism may be bonded onto the sealed second light-emitting element. Furthermore, the transparent plate that seals the second light-emitting element may also have an optical part function.

The substrate 10 may also include a reflection member (reflector) having a reflecting surface thereon to efficiently output light of the first light-emitting element and the second light-emitting element. Furthermore, a reflective coat may also be formed on an inner surface of the substrate 10 (package)/side of the light-emitting element. The reflection member/reflective coat may be disposed on both or one of the first light-emitting element and the second light-emitting element. As the reflective coat, a film of white paint, Ag, Al, Au or the like may be formed. The reflective coat formed on the reflecting surface of the first light-emitting element may be the same as or different from the reflective coat formed on the reflecting surface of the second light-emitting element.

A mixture of a plurality of types of fluorescent substances that emit light having a wavelength of 480 nm to 650 nm, longer than the light-emitting wavelength of the light-emitting element may also be used as the fluorescent substance 12A of the fluorescent resin 12.

Before mounting the second light-emitting element, a heatsink may be provided to efficiently dissipate heat generated in the second light-emitting element to the substrate. The heatsink may be a transparent member or an opaque member. In the case of the transparent member, aluminum nitride, glass, sapphire, quartz or the like may be used. Aluminum nitride having a high thermal conductivity is particularly preferable from the standpoint of improving heat dissipation characteristics. In the case of the opaque member, metal such as Cu, Al, SUS or brass may be used, but the opaque member should be prevented from significantly shielding the light emitted from the first light-emitting element and the fluorescent resin 12.

The first light-emitting element and the second light-emitting element may have a same size or different sizes. Here, the size refers to a length between sides of the top surface/under surface of the LED.

Regarding the first light-emitting element and the second light-emitting element, both may be turned on simultaneously or only one may be turned on or both may be turned on alternately.

After mounting the first light-emitting element on a first substrate or first package, a second substrate or second package mounted with the second light-emitting element may be bonded onto the first substrate or second package so that the second light-emitting element is arranged on substantially the same optical axis line O of the first light-emitting element.

The light-emitting apparatus in the present modification example can realize high luminance while maintaining high level color rendering. Furthermore, since the light-emitting element in the present modification example has a two-layer structure, the light-emitting apparatus is easy to manufacture and easy to miniaturize. Furthermore, since not many electrical connections are used, the light-emitting apparatus in the present modification example provides high reliability.

### Second Embodiment

Next, a light-emitting apparatus 1A according to a second embodiment will be described. Since the light-emitting apparatus 1A is similar to the light-emitting apparatus 1 of the first embodiment, descriptions of similar components will be omitted.

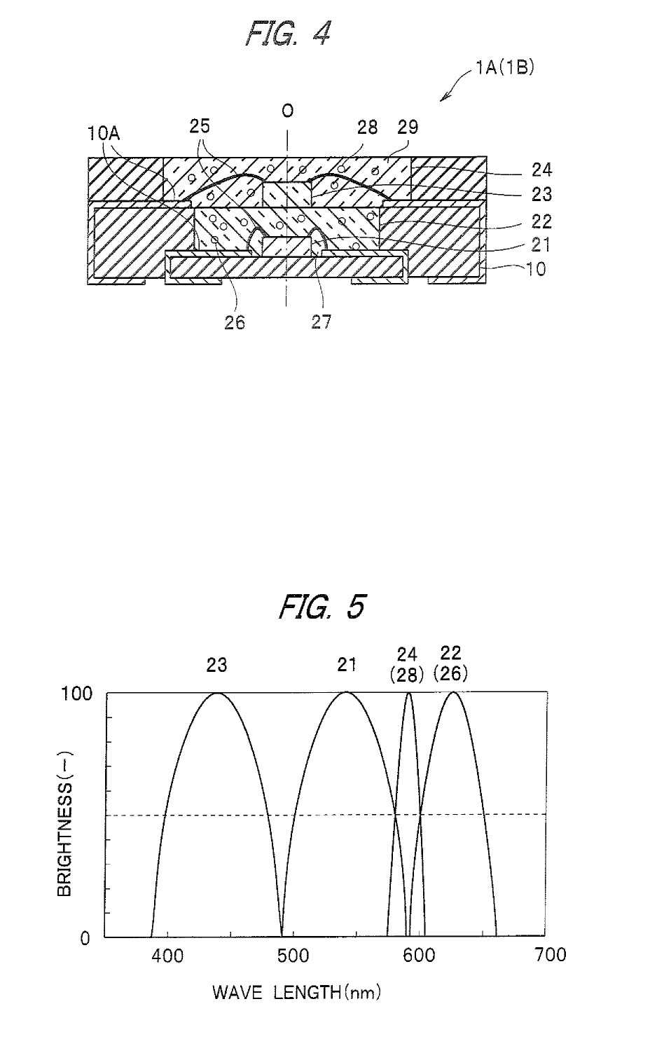

As shown in FIG. 4, the light-emitting apparatus 1A includes a blue color LED 21 which is a first light-emitting element, a first fluorescent substance mixed transparent resin (hereinafter referred to as “first fluorescent resin”) 22 which is first sealing resin, a purple color LED 23 which is a second light-emitting element and a second fluorescent substance mixed transparent resin (hereinafter referred to as “second fluorescent resin”) 24 which is second sealing resin.

The blue color LED 21 is die-bonded onto a substrate 10 and the first fluorescent resin 22 covers and seals the blue color LED 21. The purple color LED 23 is die-bonded onto the first fluorescent resin 22 and the second fluorescent resin 24 covers and seals the purple color LED 23.

The first fluorescent resin 22 is made of a transparent resin 27 mixed with a first fluorescent substance 26 and the second fluorescent resin 24 is made of a transparent resin 29 mixed with a second fluorescent substance 28.

The light-emitting wavelength of the first fluorescent substance 26 of the first fluorescent resin 22 is longer than the light-emitting wavelength of the first light-emitting element. The light-emitting wavelength of the second fluorescent substance 28 of the second fluorescent resin 24 is longer than the light-emitting wavelength(s) of both or one of the first light-emitting element and the second light-emitting element. Any combination may be adopted as long as it satisfies a relationship between the light-emitting wavelength of the fluorescent substance and the light-emitting wavelength of the light-emitting element.

For example, the light-emitting wavelengths of the first and second light-emitting elements range from 395 nm to 650 nm and the light-emitting wavelengths of the fluorescent substances of the first and a second fluorescent resin range from 450 nm to 650 nm, and a combination that satisfies the above conditions is used.

For example, the LED 21 that generates light of wavelength 500 to 580 nm (green to yellow) is sealed with the first fluorescent resin 22 mixed with the first fluorescent substance 26 that emits red light of wavelength 600 nm to 650 nm. The LED 23 that generates blue light of wavelength 395 to 480 nm on the first fluorescent resin 22 is sealed with the second fluorescent resin 24 mixed with the second fluorescent substance 28 that emits light of wavelength 580 nm to 600 nm (orange color).

In the first fluorescent substance 26 of the first fluorescent resin 22, a plurality of types of fluorescent substances that emit fluorescent light of wavelength 450 nm to 650 nm, longer than the light-emitting wavelength of the first light-emitting element may be mixed with the first transparent resin 27.

In the second fluorescent substance 28 of the second fluorescent resin 24, a plurality of types of fluorescent substances that generate fluorescent light having a wavelength longer than the light-emitting wavelength(s) of both or one of the first and second light-emitting elements may be mixed with the second transparent resin 29.

The first transparent resin 27 and the second transparent resin 29 are selected from among materials similar to those of the transparent resin 12B and 14 of the first embodiment.

As shown in FIG. 5, the light-emitting apparatus 1A generates light of a mixture of four wavelengths; light-emitting wavelength 500 nm to 580 nm of the first light-emitting element 21, light-emitting wavelength 600 nm to 650 nm of the first fluorescent substance 26 of the first fluorescent resin 22, light-emitting wavelength 395 nm to 480 nm of the second light-emitting element 23 and light-emitting wavelength 580 nm to 600 nm of the second fluorescent substance 28 of the second fluorescent resin 24. The light of a mixture of four wavelengths generated by the light-emitting apparatus 1A has fewer wavelength drops and provides high level color rendering.

That is, the light-emitting apparatus 1A has the effects of the light-emitting apparatus 1 and further provides high level color rendering.

The contents described in the modification example of the light-emitting apparatus 1 of the first embodiment are also applicable to the light-emitting apparatus 1A.

### Third Embodiment

Next, a light-emitting apparatus 1B and a medical endoscope (hereinafter referred to as “endoscope”) 2 according to a third embodiment will be described.

The light-emitting apparatus 1B has a same structure as that of the light-emitting apparatus 1A, but has different light-emitting wavelengths. That is, for example, the light-emitting apparatus 1B has a structure in which a first fluorescent resin 22 mixed with a first fluorescent substance 26 that emits light of wavelength 600 nm to 650 nm (red) seals a blue color LED 21 that generates light of wavelength 450 to 480 nm and a second fluorescent resin 24 mixed with a second fluorescent substance 28 that emits light of wavelength 580 nm to 600 nm (green) seals a purple color LED 23 that generates light of wavelength 390 nm to 445 nm located thereon.

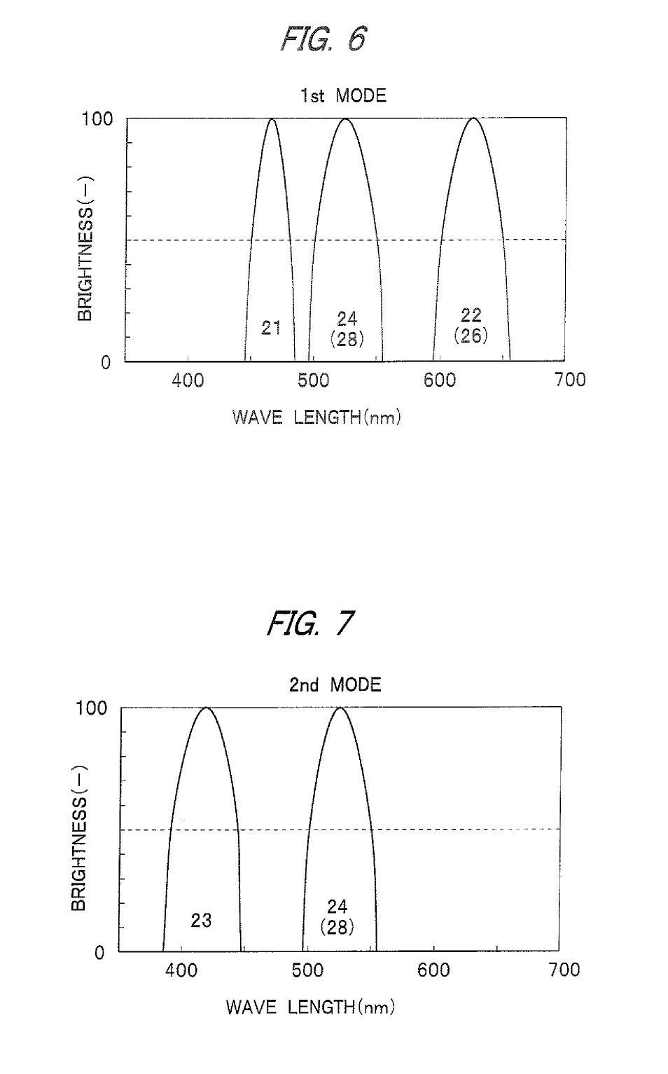

The light-emitting apparatus 1B allows a light-emitting mode to be changed (selected). That is, the light-emitting apparatus 1B passes a current only through the blue color LED 21 in a first light-emitting mode. This causes the first fluorescent resin 22 and the second fluorescent resin 24 to generate light. For this reason, as shown in FIG. 6, the light-emitting apparatus 1B generates high-luminance white color light of a mixture of three wavelengths composed of blue, red and green light in the first light-emitting mode.

On the other hand, in a second light-emitting mode, the light-emitting apparatus 1B passes a current only through the purple color LED 23. This causes the second fluorescent resin 24 to generate light. Thus, as shown in FIG. 7, the light-emitting apparatus 1B generates high-luminance narrow band light of a mixture of two wavelengths composed of purple and green light in the second light-emitting mode. Although the first fluorescent resin 22 also emits light, its light emission intensity is as small as negligible.

Here, since the light-emitting apparatus 1B is small, this is suitable for a light source apparatus disposed at a distal end portion 30A of the endoscope 2 for in-vivo illumination.

For example, as shown in FIG. 8, an illumination section 35 including the light-emitting apparatus 1B and an illumination optical system 34, and an image pickup section 33 including a C-MOS image sensor 32 and an image pickup optical system 31 are disposed at the distal end portion 30A of an insertion portion 30 of the endoscope 2. The light-emitting apparatus 1B is supplied with power via a changeover switch 36 for selectively passing a current through the blue color LED 21 or purple color LED 23.

The endoscope 2 shoots an observed image of an observation region illuminated with illuminating light emitted from the illumination section 35 using the image pickup section 33.

As observation using the endoscope 2, normal light observation (white color light observation: White Light Imaging: WLI) using visible light is widely being carried out, and moreover special light observation taking advantage of wavelength characteristics of irradiating light is also being increasingly carried out. For example, focusing attention on use of characteristics of light of being strongly absorbed by blood as well as strongly reflected/scattered by a mucous membrane surface layer, narrow band light observation (Narrow Band Imaging: NBI) radiates blue narrow band light and green narrow band light to observe blood vessels with high contrast, and can thereby highlight contrast between a capillary vessel in the mucous membrane surface layer and a thick blood vessel in a depth.

The endoscope 2 carries out narrow band light observation in the second light-emitting mode (narrow band imaging mode) of the light-emitting apparatus 1B. Furthermore, the endoscope 2 carries out normal observation in the first light-emitting mode (white light imaging mode) of the light-emitting apparatus 1B.

A deviation of an optical axis of white color light from an optical axis of narrow band light results in a difference in the way an object is seen between an observation using white color light and an observation using narrow band light, making it more difficult to observe the object. However, with the light-emitting apparatus 1B, even when the light-emitting mode is changed, a center of emitted light remains on the same optical axis line O, which makes observation easier.

The endoscope 2 having the light-emitting apparatus 1B allows switching between a white color light mode with high-level color rendering made up of light of a mixture of three waves, which is a mixture of three primary colors of light (red, green, blue), on substantially the same optical axis line O and a narrow band light mode.

The light-emitting apparatus 1B of the endoscope 2 may provide a changeover switch which makes it possible to selectively pass a current through the blue color LED 21 or purple color LED 23 so that a high-level color rendering light observation mode using a same light of a mixture of four wavelengths as that of the light-emitting apparatus 1A may be selected as a third light-emitting mode or the high-level color rendering light observation mode may be used as a white light imaging mode.

Having described the preferred embodiments of the invention referring to the accompanying drawings, it should be understood that the present invention is not limited to those precise embodiments and various changes and modifications thereof could be made by one skilled in the art without departing from the spirit or scope of the invention as defined in the appended claims.

What is claimed is:

1. A light-emitting apparatus comprising:
a first light-emitting element that generates light of a first wavelength band;
a first sealing resin that covers the first light-emitting element, the first sealing resin being made of a transparent resin mixed with a first fluorescent substance that converts the light of the first wavelength band to light of a third wavelength band having a longer wavelength;
a second light-emitting element disposed on the first sealing resin and generating light of a second wavelength band; and
a second sealing resin made of a transparent resin that covers the second light-emitting element.

2. The light-emitting apparatus according to claim 1, wherein the first light-emitting element and the second light-emitting element are LEDs.

3. The light-emitting apparatus according to claim 2, wherein the first light-emitting element and the second light-emitting element are disposed on a same optical axis line.

4. The light-emitting apparatus according to claim 3, wherein a light-emitting mode can be switched between a first light-emitting mode in which only the first light-emitting element is made to emit light and generate light of a mixture of three wavelengths and a second light-emitting mode in which only the second light-emitting element is made to emit light and generate light of a mixture of two wavelengths.

5. The light-emitting apparatus according to claim 4, wherein the light of the first wavelength band is blue light, the light of the second wavelength band is purple light, the light of the third wavelength band is red light and the light of the fourth wavelength band is green light.

6. The light-emitting apparatus according to claim 5, wherein the light-emitting apparatus is a light source for a medical endoscope, the first light-emitting mode of which is a white color light mode and the second light-emitting mode of which is a narrow band light mode.

7. The light-emitting apparatus according to claim 3, wherein the second sealing resin is mixed with a second fluorescent substance that converts the light of the second wavelength band to light of a fourth wavelength band having a longer wavelength.

8. The light-emitting apparatus according to claim 7, wherein a light-emitting mode can be switched between a first light-emitting mode in which only the first light-emitting element is made to emit light and generate light of a mixture of three wavelengths and a second light-emitting mode in which only the second light-emitting element is made to emit light and generate light of a mixture of two wavelengths.

9. The light-emitting apparatus according to claim 8, wherein light of a mixture of four wavelengths is generated from the first light-emitting element having a light-emitting wavelength of 500 nm to 580 nm, the first fluorescent substance having a light-emitting wavelength of 600 nm to 650 nm, the second light-emitting element having a light-emitting wavelength of 395 nm to 480 nm and a second fluorescent substance having a light-emitting wavelength of 580 nm to 600 nm.

10. An endoscope comprising:
an image pickup optical system;
an image sensor;
an illumination optical system;
a light-emitting apparatus comprising a blue color LED that generates blue light, a first fluorescent resin that covers the blue color LED, mixed with a first fluorescent substance that converts the blue light to red light, a second LED disposed on the first fluorescent resin and generating purple light and a second fluorescent resin that covers the purple color LED, mixed with a second fluorescent substance that converts the purple light to green light; and
a changeover switch to selectively pass a current through the blue color LED or the purple color LED.

11. The endoscope according to claim 10, wherein the blue light has a wavelength of 450 to 480 nm, the red light has a wavelength of 600 nm to 650 nm, the purple light has a wavelength of 390 nm to 445 nm and the green light has a wavelength of 580 nm to 600 nm, and
the light-emitting apparatus generates white color light of a mixture of three wavelengths made up of the blue light, the red light and the green light when a current is passed through the blue color LED and generates narrow band light of a mixture of two wavelengths made up of the purple light and the green light when a current is passed through the purple color LED.

12. The endoscope according to claim 11, wherein the first LED and the second LED are disposed on a same optical axis line.
